(19) United States
(12) Patent Application Publication
(10) Pub. No.: US 2015/0157435 A1
(43) Pub. Date: Jun. 11, 2015

(54) EQUINE FITNESS MONITOR

(71) Applicant: PegaSense, Inc., Somerville, MA (US)

(72) Inventors: Katherine Leigh Chasins, Clarkston, MI (US); Oscar Orlando Salgado, Somerville, MA (US); Rachel Ariane Dias-Carlson, Cambridge, MA (US); Viveka Mishra, Boston, MA (US); Ramya Narayana Swamy, Nashua, NH (US)

(73) Assignee: PegaSense, Inc., Somerville, MA (US)

(21) Appl. No.: 14/563,276

(22) Filed: Dec. 8, 2014

Related U.S. Application Data
(60) Provisional application No. 61/912,793, filed on Dec. 6, 2013.

(51) Int. Cl. A61D 13/00; A61B 5/11; A61B 5/024; A61B 5/01

(52) U.S. Cl. CPC A61D 13/00; A61B 5/01; A61B 5/1118; A61B 5/024

(57) ABSTRACT

Monitoring the physiological state of an equine by measuring data with which a user can make educated decisions for the well-being of his equine. The physiological monitoring system may consist of wearable sensor units that are removably attached to the equine and a display hub unit which collects data and displays the results to the user. The wearable sensor unit measures data from the equine and sends that data to the display hub unit. The display hub unit then uses that data to evaluate the physiological status of the equine. The evaluation can be based on ambient temperature, heart rate, accelerometer, and skin temperature data. These data may be manipulated by different methods in order to determine a physiological state. These methods include comparisons between bilaterally symmetric measurements, comparisons to a threshold value, changes with respect to time, and comparisons to a baseline state.

### CROSS-REFERENCE TO RELATED APPLICATIONS

The present application claims priority under 35 U.S.C. §119(e) to U.S. Provisional Patent Application Ser. No. 61/912,793, titled “Temperature sensing boots and alert device for equine sports” and filed on Dec. 6, 2013, the contents of which are incorporated herein in their entirety.

### BACKGROUND

Monitoring equine fitness and health is essential when training and competing. A monitor allows for safe and effective training so that equine's health is not compromised. Fitness information gathered about the physiological status allows for an improved understanding of the equine's experience during exercise, resulting in more effective training and a healthier equine. Any data that can be gathered to this effect allows for data-driven, better-informed decisions that can be made to improve the fitness and health of the equine.

Training for equine sports aims to push the equine to its physiological limits in order to improve fitness and overall performance. Equine training focuses on varying physical exercises in order to allow the equine to adapt and become accustomed to exertions required by each specific type of competition. These exercises may include endurance, finesse, jumping ability, and sprint performance. The nature of training is to stress the relevant aspects of the equine's physiology so that it adapts to an increasingly higher level of exertion and performance. The danger of this is that the person executing the training regime has no way to know for certain if the training is effective over time, or if in the moment they are causing the equine to overexert, overheat, or place too much stress on a particular anatomical component, thus compromising its health.

As with human athletes, heart rate can be a valuable tool for gauging exertion during training. Heart rate data can be used to indicate overexertion if the heart rate stays above a certain threshold for the equine for an abnormal period of time. This threshold can be calculated by analyzing the relationship between an equine's heart rate and its speed at that heart rate. Overexertion can be dangerous as it pushes the equine beyond its limits and injuries are more likely to occur when the equine is exhausted.

Leg injuries affect a large amount of competitive equines in the United States—20-30% are hindered in competition by a leg injury at any given time. Connective tissue injuries begin with small changes to molecular structure and worsen over time as seen with tendinitis, tenosynovitis, and desmitis. Injuries can also be incurred spontaneously during a training session. A leading cause of injury is improper or insufficient warm-up that does not allow the connective tissue to reach the effective level of elasticity associated with increased oxygen and blood flow to muscles. The current method requires a person to gauge whether the equine is warmed-up based on personal experience, and to run hands down the equine's legs if an injury is suspected to feel for heat—the first sign of a leg injury. Detection at an early stage is infrequent due to the equine's herd instinct to mask minor pain. Because of this, minor injuries are often exacerbated until they reach a much more acute state. These subclinical injuries are typically small lesions in the tissue that begin as minor pain, but worsen quickly without rest and proper care. It has been noted that these small lesions generate a small temperature increase of 1-2° C., but the human hand—the typical method of early detection—is only capable of feeling changes in temperature of 2° C. or more. For these injuries, up to 3 weeks can pass before a limp or notable edema is evident.

When injuries do occur, rehabilitation requires hand-walking while on stall rest to facilitate exercise without re-injury, and veterinarian visits in order to estimate progress through rehabilitation. After connective tissue damage, the collagen tissue that immediately replaces the damaged area has been noted to have different thermal properties from the original tissue fiber. As an injury heals, the tissue converts back to the original type and its thermal properties change accordingly. This makes it possible to quantifiably monitor rehabilitation progress.

Professional sports are increasingly adopting devices designed to monitor fitness and prevent and predict frequent injuries. These have seen high success rates in sports such as soccer and basketball where injuries for adopting teams have dropped dramatically. Personal fitness monitoring has also grown in popularity as is evident by the vast number of smart watches and fitness trackers being made. Deterred by the size, cost, and inaccessibility of current diagnostic tools for equines, wearable monitors and diagnostics have begun to reflect other professional sports and enter the equine sport industry. These tools monitor movement and other indicators of training or health, but do not directly monitor vulnerable areas or send the data through an analysis to this effect. As with the inventions created to prevent professional athlete injuries in basketball and soccer, the most effective device for the competitive equine is one that takes key factors about the sport and athlete into consideration when preventing frequent injuries or training for a specific means.

### SUMMARY

In one embodiment, there is provided an apparatus to detect a temperature of at least one anatomical feature of an equine within a lower portion of a leg of the equine. The lower portion comprises a portion of the leg of the equine from a carpus or hock of the leg to a coffin joint of the leg of the equine. The apparatus comprises a housing having a shape conforming to a shape of at least a part of the lower portion of the leg of the equine and arranged to be removably attached to the lower portion of the leg of the equine to be worn by the equine. The apparatus further comprises at least one temperature sensor integrated into the housing at one or more respective positions within the housing. Each one respective position of each one temperature sensor is a position that, when the apparatus is worn by the equine, corresponds to a position of one anatomical feature, of the at least one anatomical feature, within the lower portion of the leg of the equine and a temperature detected by the one temperature sensor is indicative of a temperature of the one anatomical feature to which the position of the one temperature sensor corresponds.

In another embodiment, there is provided an apparatus comprising at least one wireless receiver and at least one control circuit configured to receive via the at least one wireless receiver, over time, temperature information indicative of a temperature of at least one anatomical feature within a first leg of an equine, evaluate the temperature information to determine a physiological state of the at least one anatomical feature within the first leg, and output via a user interface an indication of the physiological state.

### BRIEF DESCRIPTION OF THE DRAWINGS

The accompanying drawings will be used to describe the invention and its features. Only the components in each drawing that are significant are labeled with a number that is used to refer to them. Note that nothing is drawn to scale. In the drawings:

FIG. 1 is an illustration of an example of a monitoring system being used by a rider and his/her equine;

FIG. 2 is a flowchart of how each element in the system may communicate with each other in some embodiments;

FIG. 3 is an illustration of an example of a first element or wearable measurement unit in the form of an open-front boot that may be attached to an equine's leg;

FIGS. 4A, 4B, and 4C show detailed illustrations of examples of how the wearable measurement unit may be aligned on an equine's leg;

FIG. 5 is a block diagram of a possible embodiment of the wearable measurement unit showing its components;

FIG. 6 is a flow chart of an exemplary process that the wearable measurement unit may perform;

FIG. 7 is an illustration of an example of a second element or display hub unit in the form of a watch that may be worn on the user's wrist;

FIG. 8 is a block diagram of a possible embodiment of the display hub unit showing its components;

FIG. 9 is a flow chart showing an exemplary process that the display hub unit may perform;

FIG. 10 is a flow chart showing an exemplary process for determining the physiological state of an anatomical feature during rehabilitation through comparisons to pre-injury data;

FIG. 11 is a flow chart showing an exemplary process for determining if an equine is experiencing overexertion or overheating;

FIG. 12 is a flow chart showing an exemplary process for determining the physiological state of an anatomical feature through comparison to expected measurements;

FIG. 13 is a flow chart showing an exemplary process for detecting the physiological state of an anatomical feature on one leg through comparisons to a bilaterally symmetric leg;

FIG. 14 is a flow chart showing an exemplary process for detecting the physiological state of a leg via the temperature profile and distribution;

FIG. 15 is a flow chart showing an exemplary process of determining a physiological state based on temperature change of an anatomical feature over time;

FIG. 16 is a graph showing example temperature data that was collected from an exemplary system; and

FIG. 17 is an illustration showing example temperature data and the temperature distribution in relation to the anatomical features that was collected from an exemplary system.

### DETAILED DESCRIPTION

Applicant has recognized and appreciated that the occurrence of tendon and ligament injuries in equines may be prevented and/or reduced by monitoring of physiological data. More particularly, Applicant has recognized that a variety of physiological information may be obtainable by one or more devices when worn by the equine. Such devices may include one or more sensors, including temperature sensors, positioned in proximity to locations where injuries are common among equines, such as portions of an equine's leg. A device may include a housing and the one or more sensors may be integrated into the housing. For example, the housing may have a shape that conforms to a portion of an equine's leg such that, when the device is worn by the equine, a sensor may be positioned with respect to an anatomical feature of the leg. When a temperature sensor is integrated into the housing, the respective position within the housing may correspond to a position of an anatomical feature and a temperature detected by the temperature sensor may indicate a temperature of the anatomical feature.

Further, the Applicant has recognized and appreciated that such physiological information is most useful when it is presented to a user. In some instances, the physiological information may be displayed to a user with an intuitive user interface. Additionally or alternatively, physiological information obtained from an equine may be analyzed by an apparatus to determine a physiological state of the equine and/or one or more legs of the equine. By presenting the one or more physiological states to a user, the physiological information may be easier to understand and utilize. An apparatus may receive physiological information from one or more sensors and evaluate the physiological information to determine a physiological state. For example, an apparatus may receive temperature information indicative of a temperature of an anatomical feature of an equine's leg and determine a physiological state of the anatomical feature by evaluating the temperature information. The apparatus may include a user interface and the physiological state may be outputted via the user interface.

The ability to quantifiably monitor or diagnose the physiological state of an equine's leg has been limited to non-wearable and/or static techniques, which require the equine to be stationary or at rest. These techniques include but are not limited to Magnetic Resonance Imaging (MRI), Computerized Tomography (CT), Infrared Thermography, and Ultrasound. The collected information is typically analyzed by an experienced user or professional in order to understand the equine's status.

Applicant has recognized and appreciated that such methods are disadvantageous for a variety of reasons. First, the equipment required to collect data may be difficult or impossible to move, requiring the equine to be transported to the equipment. Second, these techniques can only be used when the equine is stationary, limiting the possibility of monitoring during exercise and requiring the need to drug the equine. Finally, the information collected from these imaging techniques is qualitative and may require analysis by an experienced user in order to make conclusions about the physiological state.

Applicant has recognized and appreciated the advantages of a monitoring system that includes one or more devices having sensors that can be worn by an equine, even while the equine is active, and an apparatus that obtains data from the sensors and determines, from the data, the physiological state of the equine, and displays the physiological state and other information obtained from the sensors to the user via a user interface. For example, sensors may be used to obtain temperature information, heart rate information, and speed information and the user interface may show the physiological state in addition to information about the equine's heart rate, speed, and average body temperature. Such information obtained from the sensors may aid the user when making training and healthcare decisions for the equine during and after a ‘session,’ where ‘session’ refers to any time the system is being used and may include but is not limited to training sessions, rehabilitation sessions, turnout, and stall rest.

Embodiments may be used to combine physiological, environmental, and kinematic data collected by sensors into an output that can be communicated to the user. Described herein are various embodiments of a physiological monitoring system that analyzes physiological information from an equine and the equine's environment, and displays this analyzed physiological information to a user, such as in real time.

Some embodiments of the system may include three components, the first of which is a wearable sensor unit that is to be worn by an equine, the second a display hub unit, and the third a data visualization tool.

The first type of component of these embodiments of the system, a wearable sensor unit, may be attached to the lower portion of an equine leg by wrapping around the leg. The wearable sensor unit may cover at least half the circumference of that portion. The wearable sensor unit may consist of at least one temperature sensor that may be placed on an interior side so that it is facing the equine when the unit is attached. Additionally, the wearable sensor unit may include at least one temperature sensor on an exterior side of the wearable sensor unit to sense ambient temperature. Further, the at least one temperature sensor on the interior side may be placed so that it substantially aligns with an anatomical feature when the wearable sensor unit is attached to the equine's leg. For example, the anatomical feature may be connective tissue such as tendons or ligaments that are located in the lower portion of the equine's leg. The location of a temperature sensor may correspond to a location of an anatomical feature such that temperature information obtained by the temperature sensor is indicative of the anatomical feature. The location of the temperature sensor may also correspond to the location of an anatomical feature by not necessarily precisely aligning to the anatomical feature, but instead being placed within a diagnostically-sufficient distance from the anatomical feature such that temperature information indicative of a temperature of the anatomical feature may still be obtained. Those skilled in the art will appreciate the limits of the distance beyond which temperature information for an anatomical feature, useful for carrying out the diagnostic processes described herein, may not be reliably obtained. In some instances, a temperature sensor may be displaced within less than 0.5 inches, within less than 1 inch, and within less than 2 inches from the anatomical feature. Some anatomical features, such as connective tissues, may have long dimensions, such as tendons that extend for multiple inches or over a foot in length. In such cases, the location of the sensor may correspond to any suitable location along the anatomical feature. Applicant has recognized and appreciated, however, that it may be advantageous in embodiments or for some anatomical features to have the locations of the temperature sensors correspond to locations at which the anatomical feature is closest to an outer layer of skin of an equine. In some embodiments, the locations to which the sensors correspond may be locations of anatomical features in an average equine, without accounting for difference in conformation or between breeds of equines. In other embodiments, a wearable sensor unit may be specific to a breed of equine or to a specific equine and the locations of sensors may correspond to locations at which one or more anatomical features are closest to an outer layer of skin of an equine for an average equine of that breed or for that specific equine.

The wearable sensor unit is not limited to one temperature sensor. The wearable sensor unit may contain multiple temperature sensors positioned to align with anatomical features at locations where the anatomical features are estimated to be closest to the skin surface. The anatomical features may have just one sensor monitoring it or multiple sensors monitoring it at different locations. Multiple temperature sensors may be positioned at an outer layer of the horse's skin at locations that correspond to different positions of the same anatomical feature. Additionally, sensors may be positioned at locations that correspond to different anatomical features. Furthermore, the one or more sensors of a wearable sensor unit are not limited to temperature sensors as the wearable sensor unit may contain other types of sensors. These types of sensors may include:
Heart rate sensor
Accelerometer
Position sensor
Lactic acid sensor
Environmental sensors such as ambient temperature and humidity
Other potential physiological sensors

The wearable sensor unit may include at least one processor that can process the data acquired by the sensors described above. The wearable sensor unit may further include at least one wireless transmitter that may transmit the processed data to the second type of component of the system, the display hub unit, for analysis. Additionally, the wearable sensor unit may include a power source which powers the at least one processor, the at least one wireless transmitter, and the sensors described above. These components and sensors are connected together by a functioning and suitable control circuit.

In a first embodiment, the wearable sensor unit may take on the form of a traditional equine boot. Examples of these are splint boots, open-front boots, and ankle boots. The wearable sensor unit may be configured for either a fore leg or a hind leg of an equine. These types of boots are generally used for impact protection or support for connective tissues in the lower leg. Therefore, the first embodiment is to be constructed such that the parts and circuit described above are protected from potential damage or from incurring any physical damage to the equine. This embodiment may be attached to the lower portion of an equine's leg such that it may be removed, and when worn it may cover any suitable amount of the leg, including more than half of a lower portion of the leg of the equine. This may be done with Velcro, straps, or stud buttons (also known as snap fasteners). Either of these methods may be implemented with conductive materials such that they may be used as a switch for the circuit that is described above.

In a second embodiment, the wearable sensor unit may take on the form of a traditional equine leg wrap. Wraps are generally used for connective tissue support. These wraps are applied by wrapping multiple times around the equine's leg at consistent tension. The wrap may be held intact by Velcro. The wrap is to be constructed such that applying the wrap correctly is evident for the user. Applying the wrap correctly means orienting the wrap such that when attached to a portion in the lower leg of the equine the sensors are aligned to the corresponding anatomical features. The Velcro attachment may be implemented with conductive Velcro so that it may be used as a switch.

In a third embodiment, the wearable sensor unit may take the form of an insert that may be placed between a portion of an equine's lower leg and existing equine leg protection equipment. The existing equine leg protection equipment may be any of the previously mentioned such as splint boot, leg wrap, etc.

The second type of component in these embodiments of the physiological monitoring system, the display hub unit, has four main functions: receiving data, storing data, analysis of the data, and a real-time communication of the results of the analysis to the user. The device is comprised of at least a microcontroller, a wireless transmitter, a power circuit, a data storage medium, and a user interface. The user interface may be designed to communicate any or all information pertaining to the physiological state of the equine to the user. An example of the user interface is a visual display. The display may consist of a screen, a virtual projection such as a heads up display, a set of one or more LED's, or any other indicator lights. Other examples of user interfaces provide auditory and/or tactile signals, including but not limited to a vibration, pulse, buzz, or speakers, which can be used on their own or in addition to the visual display.

In a first embodiment, the display hub unit may take the form of a watch. In this form, it may be worn on the user's wrist for quick glances and easy user interaction. In a second embodiment, the display hub unit may be mounted on the equine or equipment on the equine such that the user may glance at it and may interact with it. In a third embodiment, the display hub unit may take the form of a smart phone or tablet which is carried by the user. In this last embodiment, the user may be the rider of the equine wearing at least one wearable sensor unit or a person overseeing the rider and equine.

Below is an explanation of the functions of the display hub unit. The information communicated by the display hub unit's user interface may include one of the following physiological states:
Not yet warmed up
Warmed up
Cooled down
Danger of overexertion
Overheating
Significant abnormality
Potential injury or re-injury detected and connective tissue of concern
Inflammatory response
Tissue damage restoration estimate

The first function of the display hub unit is to receive data sent from at least one wearable sensor unit via any suitable wireless manner. Potential wireless embodiments of this system may include but are not limited to; Bluetooth, ZigBee, and Radio Frequency.

The second function is to store the data. All of the data sent by the at least one wearable sensor unit and received by the display hub unit are stored in internal storage such as flash memory which may be accessed at a later time.

The third function of the display hub unit is to analyze the data in order to conclude the physiological state of the equine at any point in time. This is done using software programs loaded to the system microcontroller, historical data, and database values. The analysis is described further below.

The fourth and final function of the display hub unit is to communicate the physiological state at any given point in time to the user. The display hub unit may have at least one component that the user can operate. This component may be a button, touch screen, voice recognition system, eye tracker, or other method. The physiological state communicated via the user interface may or may not be a simplified version of the states described above in order to facilitate quick user understanding. For example, information immediately available to the rider may be one of three or more states such as ‘not yet warmed up,’ warmed up,' and ‘potentially injured.’ The rider may then have the option to interact with the interface in order to learn more details about the physiological state at any point in time. These details, which may pertain to one or more specific legs and/or one or more connective tissues, may include more descriptive and/or detailed summaries of the physiological state, information about potential injuries, and quantitative information corresponding to the sensor values or severity of alert.

The analysis software may estimate the physiological state of the equine and the display hub unit may communicate this information to the rider via one or more user interfaces. As it may be important in some embodiments for this information to be available to a rider as quickly as possible, the analysis may occur continuously whenever the system is in use so that the user may see the result within a short time such as two minutes. However, it should be appreciated that embodiments are not so limited. The display hub unit receives data from at least one wearable sensor unit, which may include one or more temperature sensors, a heart rate monitor, one or more accelerometers, or other physiological sensors in each wearable sensor unit. From this point on, the term ‘sensors’ will be used to refer to any or all types of sensors unless one type of sensor is specifically named. The information from each of these sensors is analyzed with respect to one or more of a range of criteria to determine the physiological state of the equine's legs at a given point in time.

As part of determining the change in physiological state corresponding to abnormal or dangerous health issues, in some embodiments the system uses fixed temperature thresholds for each physiological state. In other embodiments, however, the system may establish what sensor values correlate to the normal or baseline state in which an equine is healthy, unstressed, and at rest, and use these values as part of determining the changes in physiological state. These values determined for an individual equine, or for all equines in general, will hereafter be referred to as the “baseline values”. In one embodiment a profile may be created for each individual equine when the system is first used. The software may calibrate by storing sensor values for that particular equine when healthy and at rest, as determined by an experienced veterinarian as the baseline values. In a second embodiment, baseline values may be preloaded as part of the software. These values may be preloaded into the software and could be derived from clinical research studies, a database of baseline values, or a veterinarian. There may be several sets of baseline values, and the set used may be selected based on specific characteristics of the equine, such as age, gender, breed, and injury history. The set would be chosen to correspond to the characteristics input by the user. In a third embodiment, sensor values during the first few times the system may be saved in a data log, and average values of the sensor values may be used to set baseline values for the equine. In a fourth embodiment, baseline values may be the result of functions which output an expected baseline value for each use after taking into account the loaded baseline values and inputs such as ambient temperature or humidity. In a fifth embodiment, any combination of the first, second, third, and fourth embodiments may be used to establish baseline values. These baseline values may be used as part of the analysis algorithm through comparison with measured temperature values at any point in time. This comparison may determine if the physiological state deviates from the normal healthy state.

The software may also include threshold values or functions, which correspond to specific decision making processes in the algorithm. For example, a threshold value may be set which corresponds to the temperature at which tendon cells denature. This threshold value would then be compared to temperature sensor values, and if the temperature is found to be above this threshold, an abnormality may be present. Threshold values may also correspond to warmed-up temperature, cooled-down temperature, inflammatory response, maximum heart beat, etc. These threshold values may be predefined and loaded directly into the software, they may be set by a veterinarian or user, or they may be created as a function of the baseline values.

Once the baseline values have been established in the software, the algorithm proceeds to analyze data obtained from sensors of the wearable sensor unit to conclude the physiological state of one or more equine legs. The first family of physiological states concerns with the readiness of an equine for intense exercise and include but are not limited to; not yet warmed up, warmed up, and cooled down, in which ‘cooled down’ refers to a state of recovery after exercising, ‘not yet warmed up’ refers to a state in which the muscles and connective tissues do not have sufficient blood flow and/or high enough temperature to proceed to intense exercise, and ‘warmed up’ refers to a state where blood flow and/or tissue temperature is adequate to proceed to intense exercise. A state of readiness of each leg is obtained by analyzing sensor data to determine if the sensor data meets part or all of a set of criteria defining a specific state of readiness. These criteria may include but are not limited to: rate of change of skin temperature, absolute skin temperature, difference between skin temperature and ambient temperature, temperature distribution in the leg, change in temperature from resting state, heart rate value, time and distance travelled, average speed, etc.

The second family of physiological states concerns the potential presence of an injury, re- injury, or inflammation. This state may be characterized by an abnormality or sensor values corresponding to preloaded injury patterns. To determine the presence of the ‘injured’ or ‘inflamed’ physiological state, the software may analyze sensor data by determining if the sensor data meet some or all of the criteria defining an ‘injured’ or ‘inflamed’ physiological state. Analysis criteria may include but are not limited to: absolute skin temperature, difference between skin temperature and outside temperature, temperature distribution within the leg, difference between absolute temperature at corresponding locations on bilaterally symmetric legs, difference between current temperature and historical or threshold temperatures, rate of change of temperature, heart rate, and time spent at or above a particular heart rate.

The third family of physiological states concerns the potential for overexertion and/or overheating in the equine. To determine whether such a physiological state has been reached the software may analyze data from temperature sensors, a heart rate sensor, and an accelerometer both as the data is received and in relation to the immediate history such as from the same training session. Potential indicators of the physiological states ‘overexerted’ and ‘overheated’ may include but are not limited to; absolute skin temperature, difference between skin temperature and outside temperature, average training speed, time spent travelling at a particular speed, total distance travelled, time spent exercising, heart rate, and time spent at or above a particular heart rate.

Another family of physiological states concerns a status of the leg and its associated connective tissues immediately following an injury and during a recovery phase after the injury. The classification of these states may include but is not limited to: inflammation present, inflammation gone and recovery started, percentage of recovery achieved, and estimated time remaining to full recovery. In order to determine the presence, absence, and/or degree of inflammation the software may analyze the absolute temperature, temperature distribution within the leg, comparison of temperature values at the same sensor locations on different legs, temperature difference from baseline, temperature difference from historical data, rate of change of temperature, heart rate, difference between current and baseline heart rate. The analysis of the sensor data carried out in the display hub unit may be done both as the sensor data is received and any time after the sensor data has been obtained with one or more wearable sensor units.

The third component of the physiological monitoring system in these embodiments is a data visualization tool. This may be implemented as a web application on an external computing device such as a personal computer. This tool allows for the creation of a user profile for the equine, where data stored by the second component, the display hub unit, may be uploaded, saved, and attributed to that equine. The upload method may be through wireless communication such as Bluetooth, Radio Frequency, ANT, other wireless method, or through a wired connection such as a universal serial bus. Once uploaded, the data is analyzed by similar software present in the display hub unit. The results are then displayed using a user interface which may be similar to that of the of the display hub unit on a display such as a computer monitor. The data visualization tool may connect to the cloud or another internet storage system, where the data and user inputs may be saved. These user inputs may include but are not limited to: equine's behavior, overall performance, treatment given, and notes on training session intensity. The data visualization tool may also include a way for a veterinarian or other qualified professional to view the data and make recommendations to the user for care, treatment, or training of the equine.

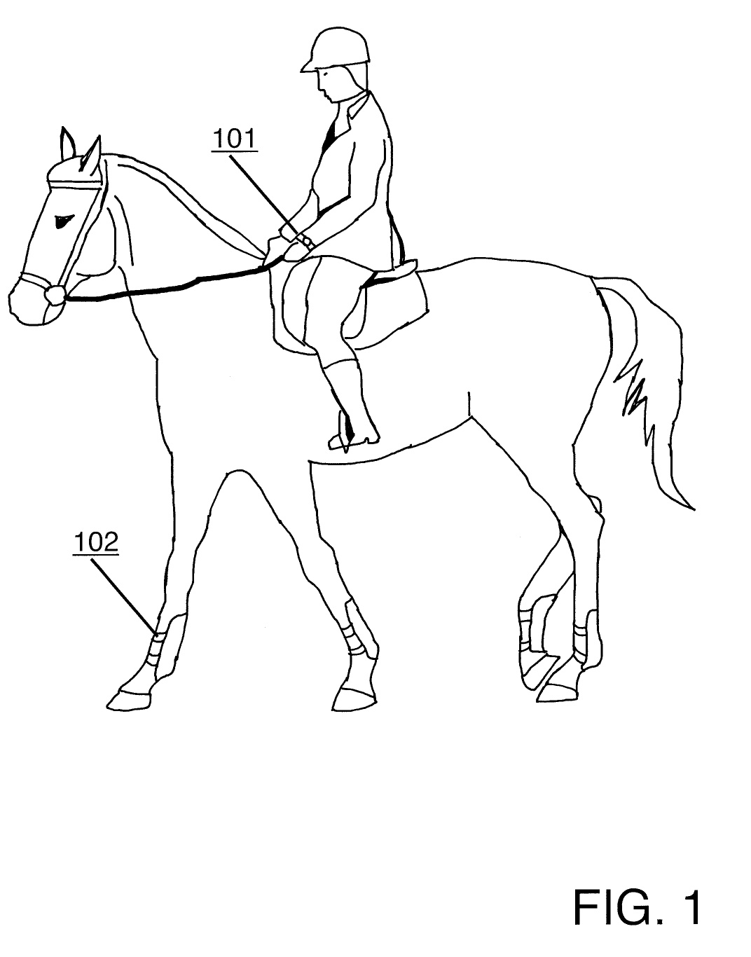

An example of the system is described below with references to FIGS. 1-16. It should be noted that the system is not limited to the example below but the features of various embodiments of the system may be understood using the example below.

FIG. 1 shows an example of the system in use. In this exemplary use of the system, the user is riding an equine during training. The equine is being monitored using the wearable sensor units 102 on its legs and the user can check on the status of the monitoring via the display hub unit 101. The wearable sensor units 102, in this case typical equine boots, communicate with the display hub unit 101, in this case a wrist device, via a wireless signal. The wearable sensor unit 102 may be any device that can be attached to the lower leg of an equine and the display hub unit 101 may be any device that can stay in range of the wireless signal that the wearable sensor units 102 emit. It may be preferred that the display hub unit 101 also be accessible to the user when the user is riding the equine. In this example, this is the case as the user is riding the equine and can access the display hub unit 101 while riding.

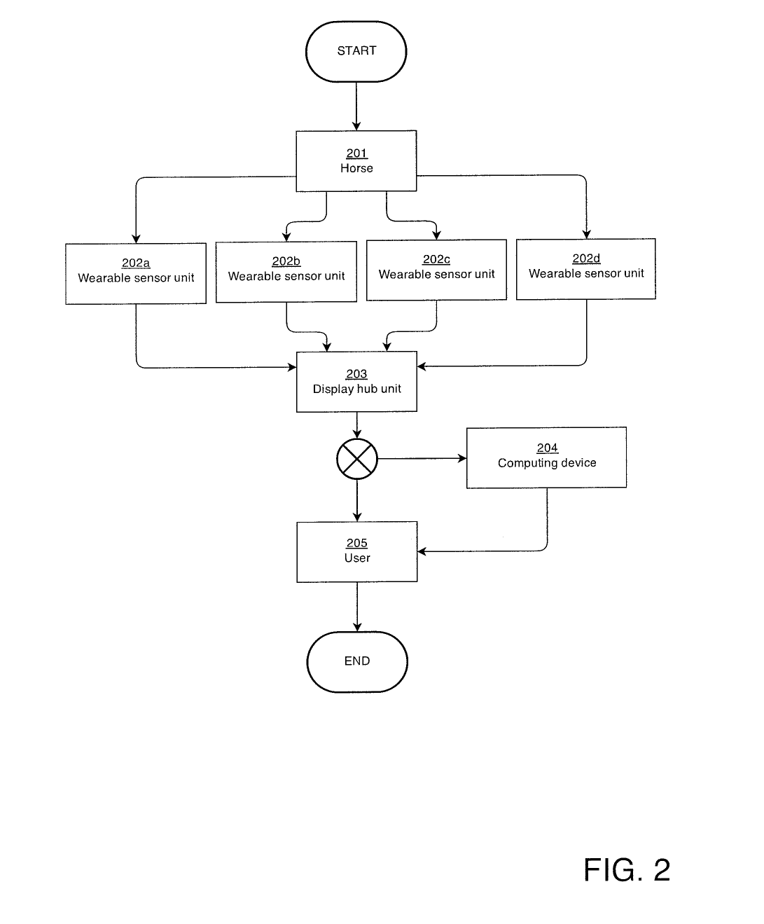

FIG. 2 shows how components in the system may relate to each other in some embodiments. The equine 201 may wear one or more wearable sensor units 202a-d which have been described above. In this example, there are four wearable sensor units 202a-d corresponding to each leg of the equine 201, however any suitable number of wearable sensor units may be worn by the equine at any given time. The wearable sensor units 202a-d all connect wirelessly to a display hub unit 203 which records the collected data. The display hub unit 203 may be worn by the user 205 and the user 205 may see an indication of the physiological state determined by analyzing of sensor data that occurs in the display hub unit 203. Analysis of sensor data to determine a physiological state is described in detail later. The display hub unit 203 may also be connected wirelessly or wired to an external computing device 204. The computing device 204 may then download data recorded by display hub unit 203, process the data, and display an output of the data to the user 205. This processing is also described in detail later. This connection between display hub unit 203 and computing device 204 may be done regardless if the display hub unit 203 is worn by the user 205 or not.

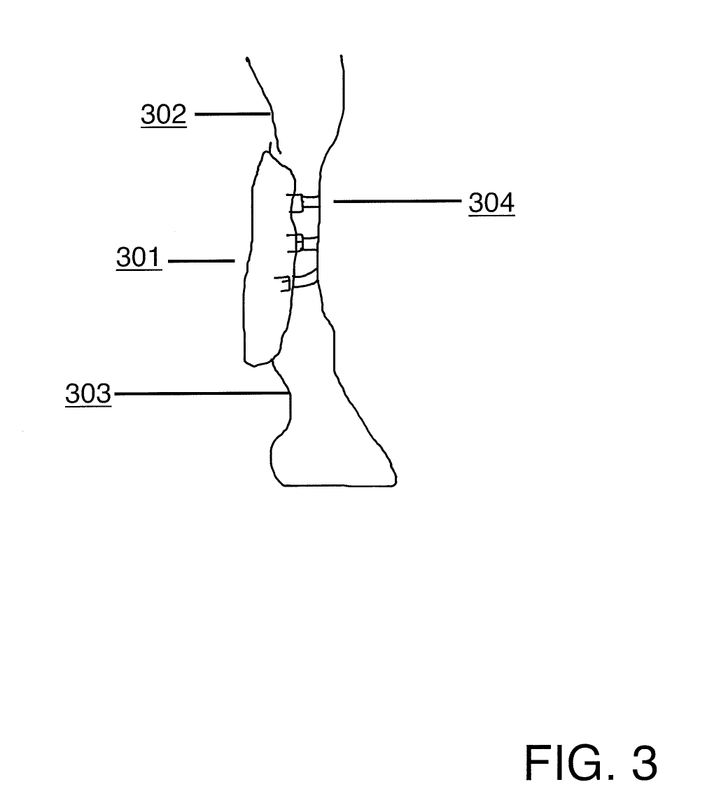

FIG. 3 shows an exemplary wearable sensor unit 301. In this example, the wearable sensor unit is in the form of an open-front boot and may be attached to a portion of the lower leg of the equine. The lower leg is defined as between the carpus or hock 302 and the coffin joint 303. The wearable sensor unit 301 may be attached by wrapping around and covering at least half of the area of the leg. In this example, the wearable sensor unit 301 is held in place with Velcro straps 304 so that the wearable sensor unit 301 may be removed and reattached with ease.

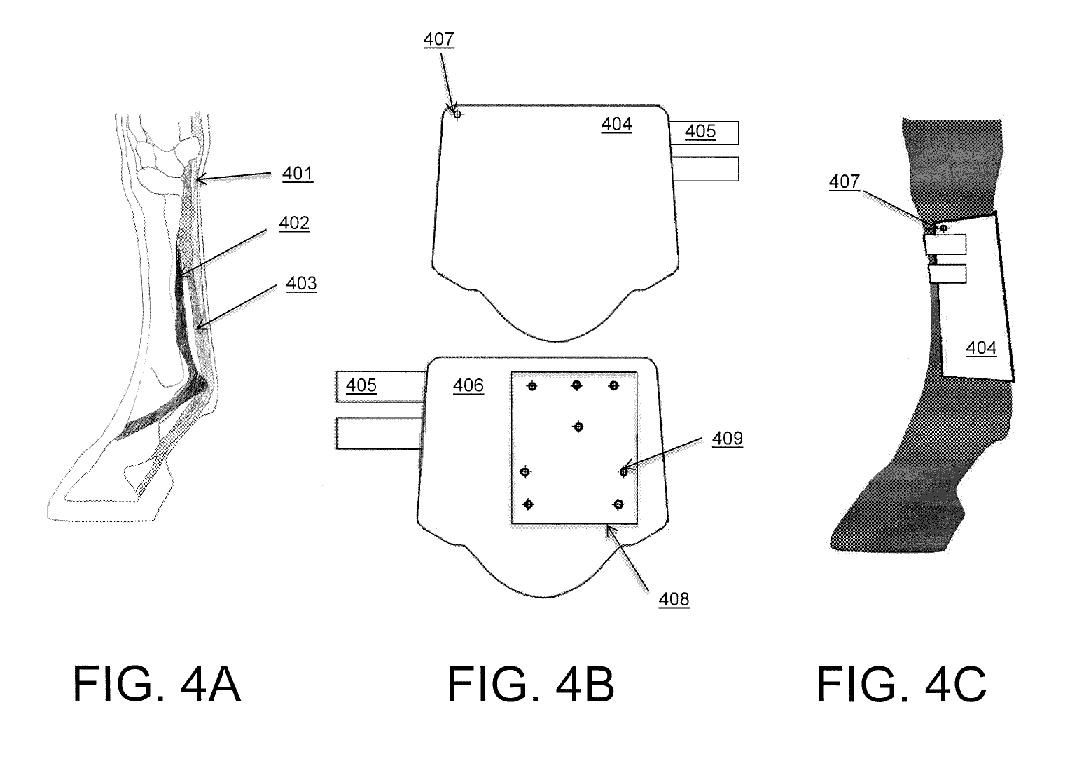

FIGS. 4A, 4B, and 4C describe examples of how the wearable sensor unit may be aligned when being attached to the equine leg. The wearable sensor unit consists of an array 408 of individual sensors 409 that are installed on the interior 406 of the wearable sensor unit. In this example, the sensors 409 are all temperature sensors but it should be noted that they may be any sensor that is mentioned above. The array 408 may be structured in any way so that the sensors 409, when the wearable sensor unit is wrapped and attached by the straps 405, correspond with anatomical features of the leg. In this example, the anatomical features include the superficial digital flexor tendon 401, the suspensory ligament 402, and the deep digital flexor tendon 403. The sensors 409 may correspond to positions that align or substantially align (as discussed above) to these three anatomical features at different locations along the lengths of the anatomical features, which may be or include locations at which one or more of the features are closest to an outer layer of skin of the equine. Finally, the wearable sensor unit may also consist of sensors that are installed on the exterior 404 of the wearable sensor unit. In this example, there is a temperature sensor 407 on the exterior so that it may measure the ambient temperature.

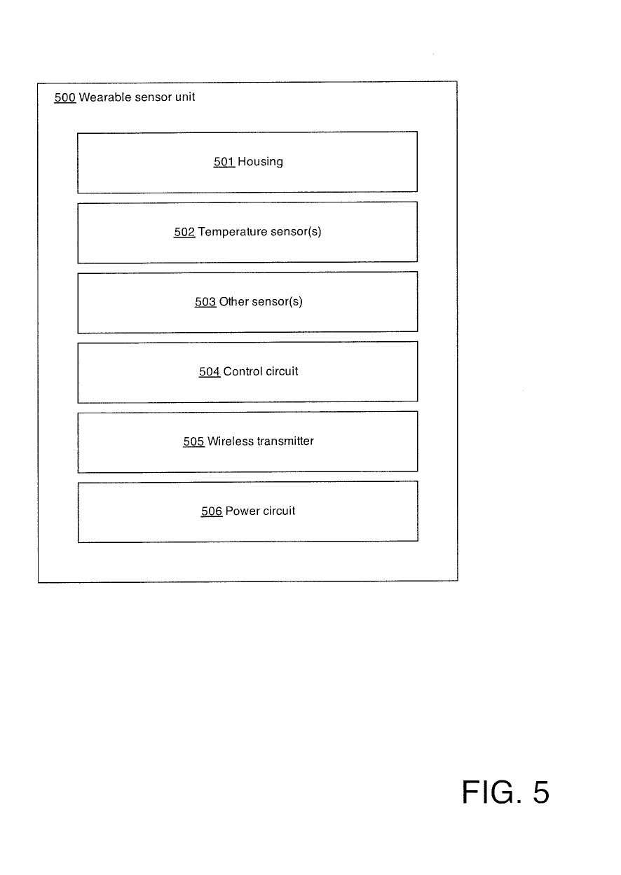

FIG. 5 shows more details of an example of the wearable sensor unit 500. As described above, the wearable sensor unit 500 consists of at least one temperature sensor 502 and other sensors 503. In this example, there is a plurality of temperature sensors 502. Additional sensors besides temperature sensors may be included in a wearable sensor unit. The wearable sensor unit 500 includes a housing 501 for the sensors and other electronics to be contained and placed correctly. In this example, the housing 501 is in a shape of an open-front boot. The wearable sensor unit 500 further includes a control circuit 504 in which all sensor data are collected. The control circuit 504 may process the data in order to transmit the data to the display hub unit. Such processing by control circuit 504 may include organizing and/or compressing the data into a suitable format for transmission by wireless transmitter 505 to a display hub unit. Wireless transmitter 505 is configured for wireless communication to the display hub unit. The wireless transmitter 505 may be a Bluetooth low energy chip. Finally, the wearable sensor unit 500 has a power circuit 506 that is used for powering the temperature sensors 502, other sensors 503, control circuit 504, and wireless transmitter 505. The power circuit 506 may consist of a battery and a regulator.

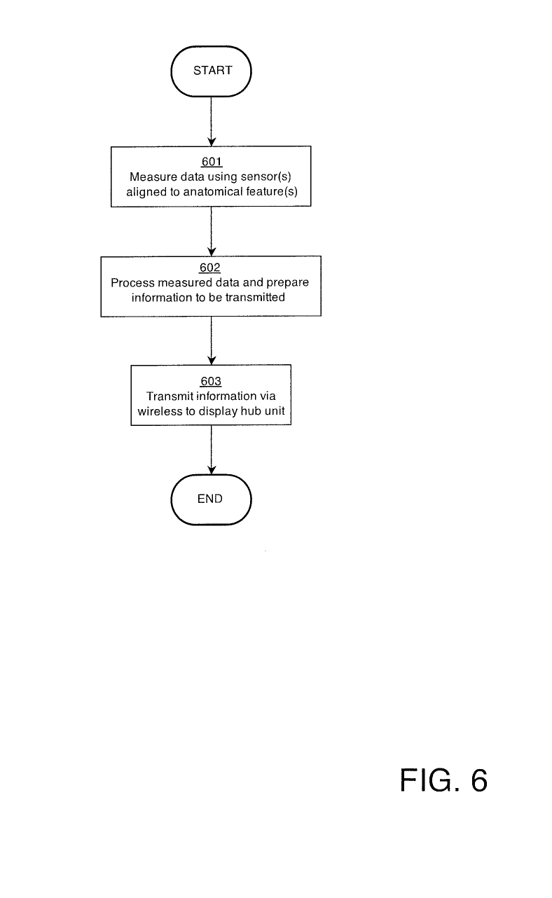

FIG. 6 shows an example of a process a wearable sensor unit may implement when in use. In block 601 the sensors measure data that correspond to the anatomical feature that the sensors coincide with or to the environment. For example, in the wearable sensor unit shown in FIGS. 4A, 4B, and 4C, the sensors in array 408 measure the temperature of the respective anatomical features at different locations and the sensor 407 measures the ambient temperature. In block 602 the control circuit processes the measured data. This processing may be simple organizing and assigning a time stamp to each measurement. In block 603, the processed data is sent using the wireless transmitter to the display hub unit. This marks the end of the process performed by the wearable sensor unit. It is repeated at a predetermined interval.

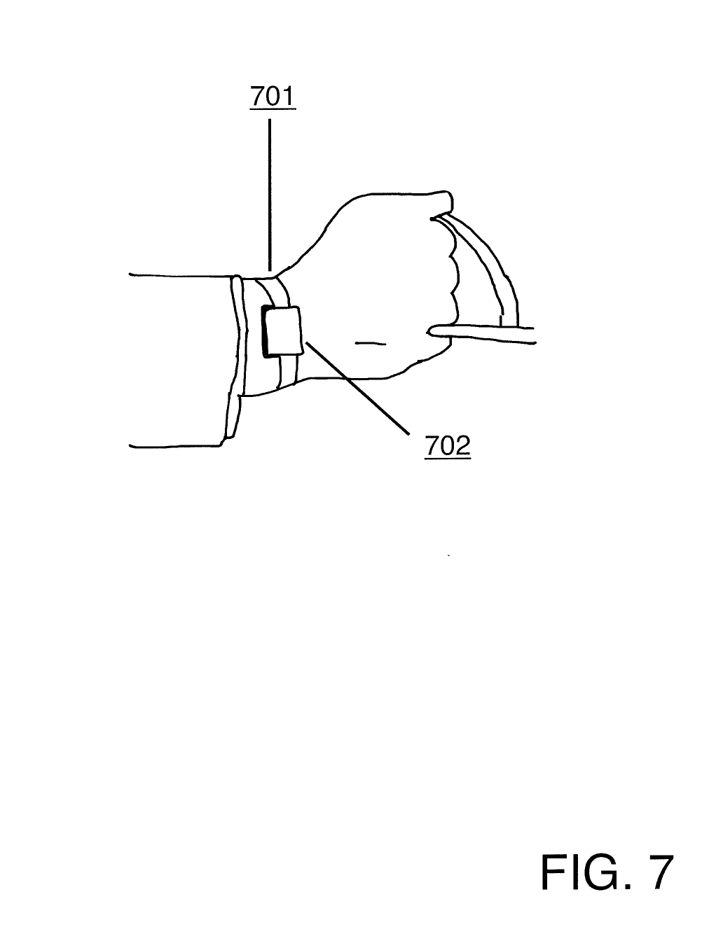

FIG. 7 shows an embodiment 701 of the display hub unit. In this example, the display hub unit 701 is located on the user's wrist and has a form of a typical watch. The display 702 may show the most important and time sensitive information to the user with a quick glance.

Alternatively, in other embodiments where the display hub unit 701 is not on the user the display 702 may be bigger and therefore may show more detailed information.

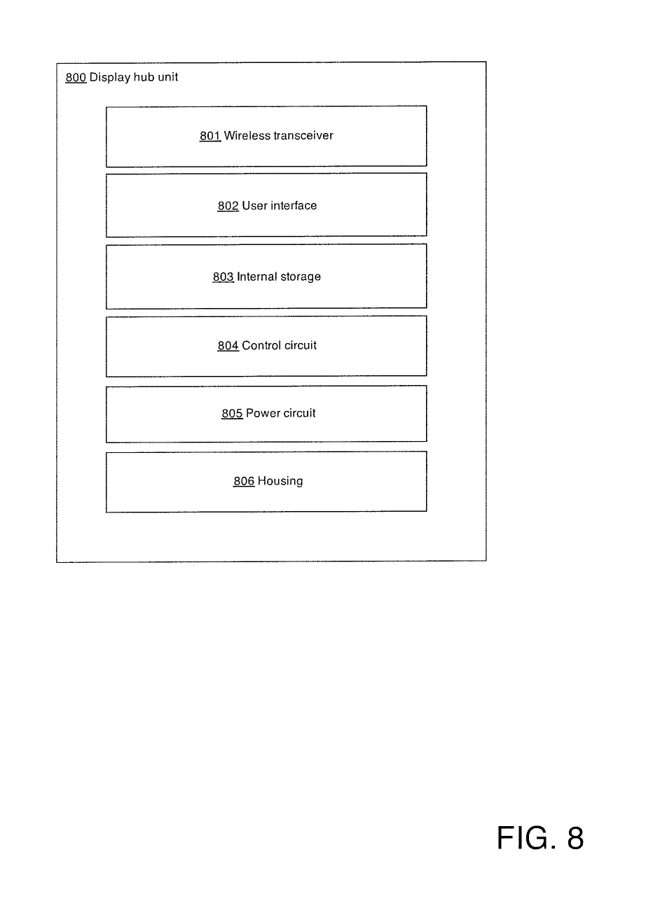

FIG. 8 illustrates the components inside an example of the display hub unit 800. A wireless transceiver 801 may receive information from the wearable sensor units. In addition, wireless transceiver 801 may also have the ability to send data to another device wirelessly. An example of the wireless transceiver 801 is a Bluetooth low energy chip. The user interface 802 is used to convey information to the user. In the example of FIG. 7 the user interface 802 is the display 702 including any drivers needed to control it. Internal storage 803 may be used to store the data that the display hub unit 800 receives from the wearable sensor unit and to store the programs that the control circuit 804 uses. The internal storage 803 may be flash memory and the amount is predetermined. The power circuit 805 may power the wireless transceiver, user interface, internal storage, and control circuit. The power circuit 805 may consist of a battery and a regulator. Finally, the housing 806 is used to contain the electronics and, if necessary, to attach to the user.

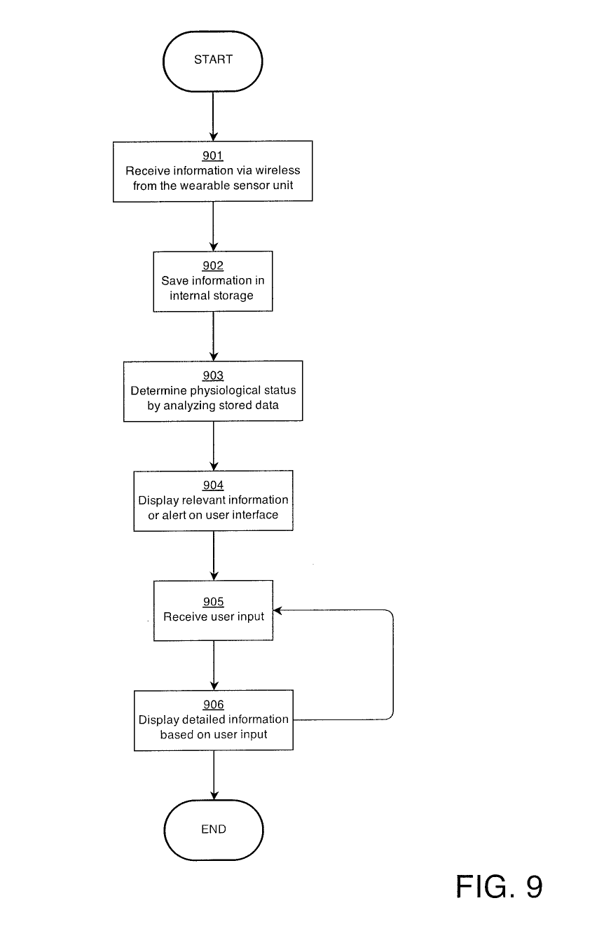

FIG. 9 shows an example of a process the display hub unit may implement when in use. This process starts in block 901 when the display hub unit receives data from the wearable sensor unit. The control circuit is configured so that in block 902 the incoming data is saved to the internal storage. In block 903 the control circuit then analyzes the stored data in order to determine a physiological status. The methods used to determine a physiological status are explained later. Once the physiological status is determined, in block 904 relevant information and/or a notification of a physiological state change is displayed on the user interface. Block 905 represents the instance any time the user interacts with the display hub unit such as a button press or a press on a touchscreen. After the user input is received, in block 906 the requested information is displayed on the user interface. This process may happen every time data is received from the wearable sensor units. Blocks 901-903 specifically may only happen when data is received while blocks 904-906 may happen in between the intervals in which data is received.

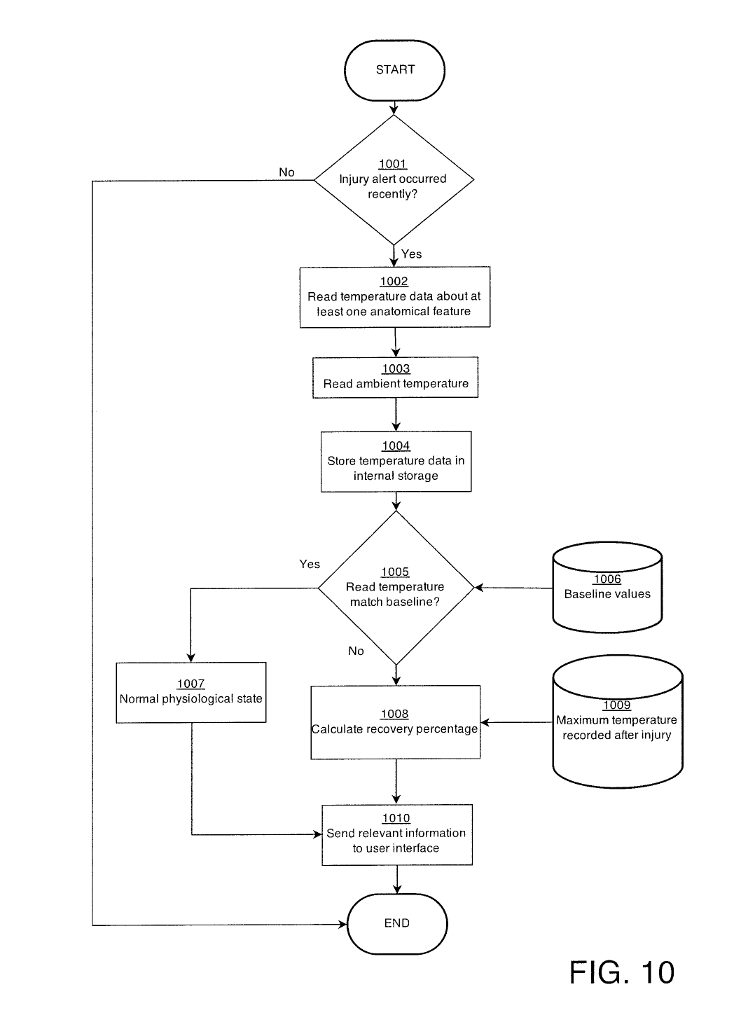

FIG. 10 shows an example of a process carried out by the display hub unit to determine the physiological state of the equine in relation to recovery progress after an injury. First, in block 1001 the system determines if an injury has recently occurred. If it has not then this process does nothing and ends. If it has, in block 1002 the temperature of the at least one anatomical feature from the most recently received group of data may be read and in block 1003 ambient temperature from the most recently received group of data may be read. These measurement data are saved to the internal storage in block 1004. Then, in block 1005 the temperature data are compared to “baseline values” 1006 which are calculated for that equine as explained above. If the read temperatures match the baseline values then in block 1007 the physiological state is determined as “normal”, i.e. the equine has fully recovered from injury. Then in block 1010 the relevant information may be sent to user interface for display. If the read temperatures do not match the baseline values, then in block 1008 the percent recovery may be calculated using the difference from the maximum temperature recorded after the injury 1009, the read temperatures, and the baseline values. This information may be sent to the user interface in block 1010 for display.

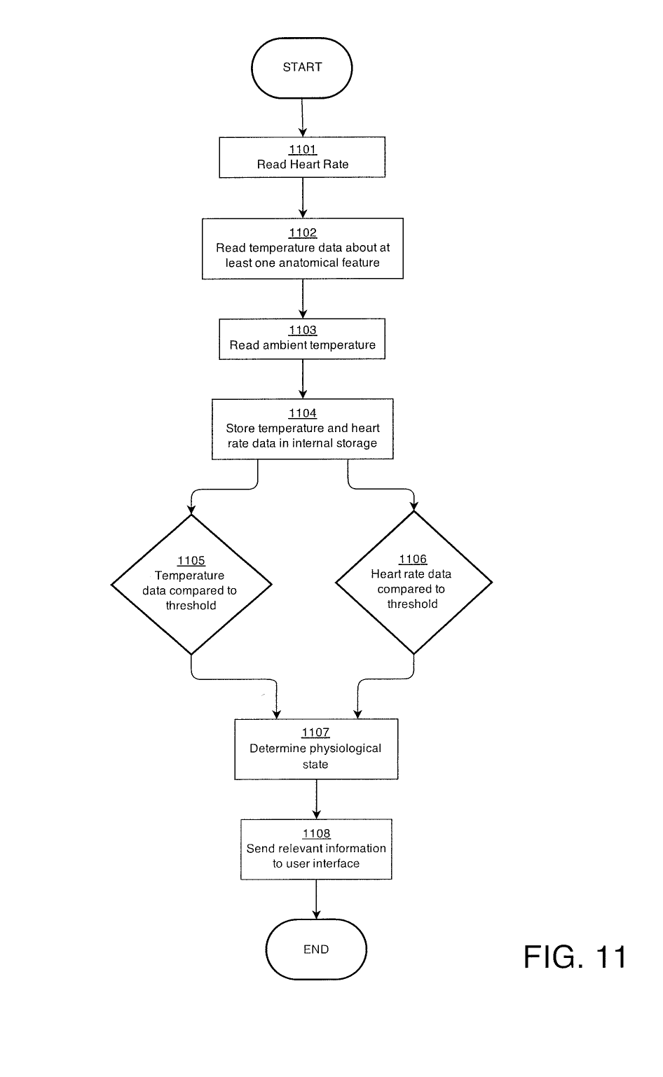

FIG. 11 shows an example of a process carried out by the display hub unit to determine if the equine is overexerted or overheated. In blocks 1101, 1102, and 1103 the heart rate, the temperature of the at least one anatomical feature, and the temperature of the environment, respectively, from the most recently received group of data may be read. These data are stored in bock 1104. In block 1105, the temperature data may be compared to a threshold, which is determined in a manner that has been described above. In block 1106, the heart rate may also be compared to a corresponding threshold. Additionally, the time spent at a heart rate above the threshold is calculated. In block 1107, the physiological state is determined as follows: if neither the read temperature is above the temperature threshold nor the read heart rate has been above the heart rate threshold for longer than a predetermined time, then the physiological state may be determined as “normal”. However, if either of those two is true then the physiological state may be determined as “abnormal”. Specifically, if the read temperature is above the temperature threshold, the physiological state may be “overheated” and if the read heart rate has been above the heart rate threshold for longer than a predetermined time, the physiological state may be “overexerted”. If both are true, the physiological state may be “overheated and overexerted”. The resulting physiological state and appropriate details are sent to the user interface in block 1108.

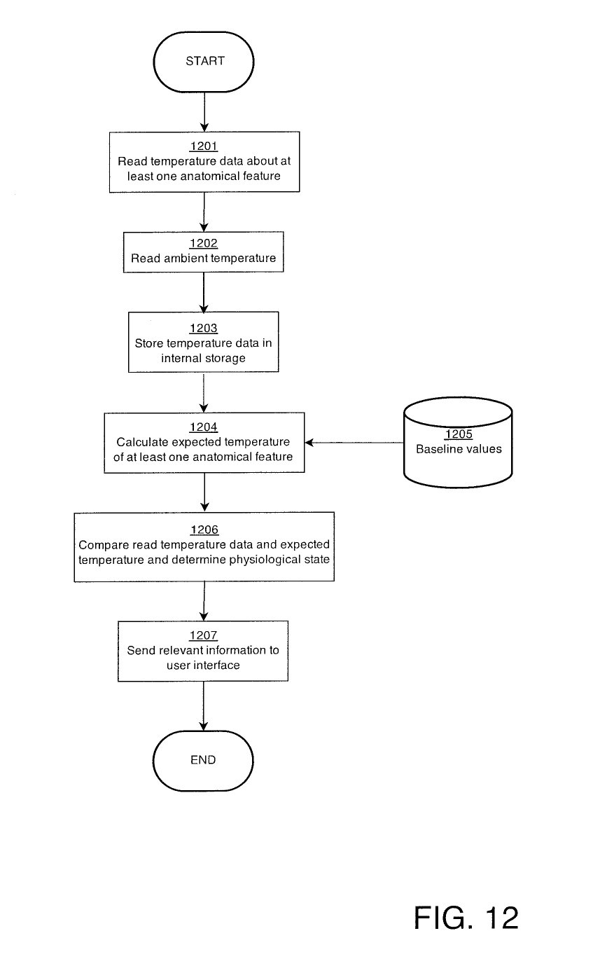

FIG. 12 shows an example of a process carried out by the display hub unit to determine the physiological state of an anatomical feature by finding the difference between the temperature measured by a sensor and the expected temperature of the corresponding anatomical feature. First, in block 1201, the temperature data from a sensor corresponding to the at least one anatomical feature from the most recently received group of data may be read. Then, in block 1202 the ambient temperature from the most recently received group of data may be read. These data are stored in block 1203. In block 1204, the expected temperature of the corresponding anatomical features may be calculated. A predefined function that includes the ambient temperature and the baseline values 1205 may be used to calculate the expected temperature. In block 1206, the physiological state may be determined by comparing the read temperature and the expected temperature. The comparison may determine whether the read temperature lies between diagnostically accepted deviations from the expected temperature. If the temperate does lie within a range based on deviations from the expected temperature, then the physiological state may be determined to be “normal”. If the temperature lies outside the range, then the physiological state may be determined to be “abnormal”. Finally, in block 1207 the determined physiological state and relevant information are sent to the user interface.

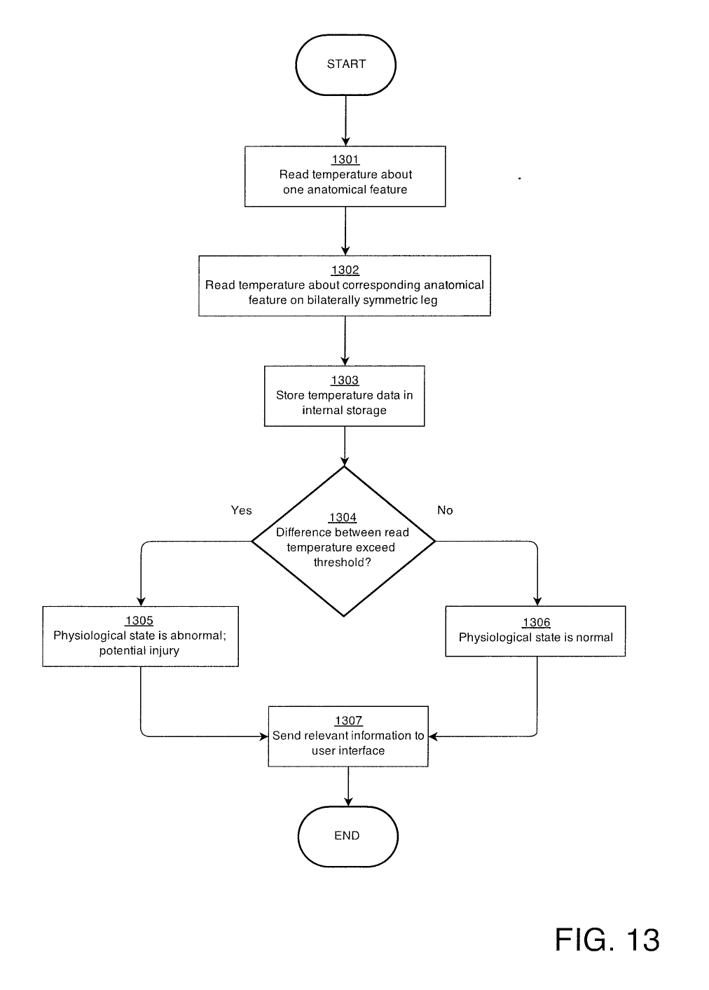

FIG. 13 shows an example of a process carried out by the display hub unit to determine whether or not a significant temperature difference at an anatomical feature exists between bilaterally symmetric legs, which may signal a potential injury. In block 1301, the temperature about an anatomical feature is read from the most recently received group of data. In block 1302, the temperature about the corresponding anatomical feature on the bilaterally symmetric leg may be read from the most recently received group of data. Then both data are stored in the internal storage in block 1303. The difference between the temperatures of the bilaterally symmetric legs may be determined in block 1304 and compared to the threshold value, which is determined as described above. If the difference does exceed the threshold, the physiological state may be determined as “abnormal” and/or “potential injury” in block 1305. If the difference does not exceed the threshold the physiological state may be determined as “normal” in block 1306. In block 1307, the determined physiological state and relevant information may be sent to the user interface.

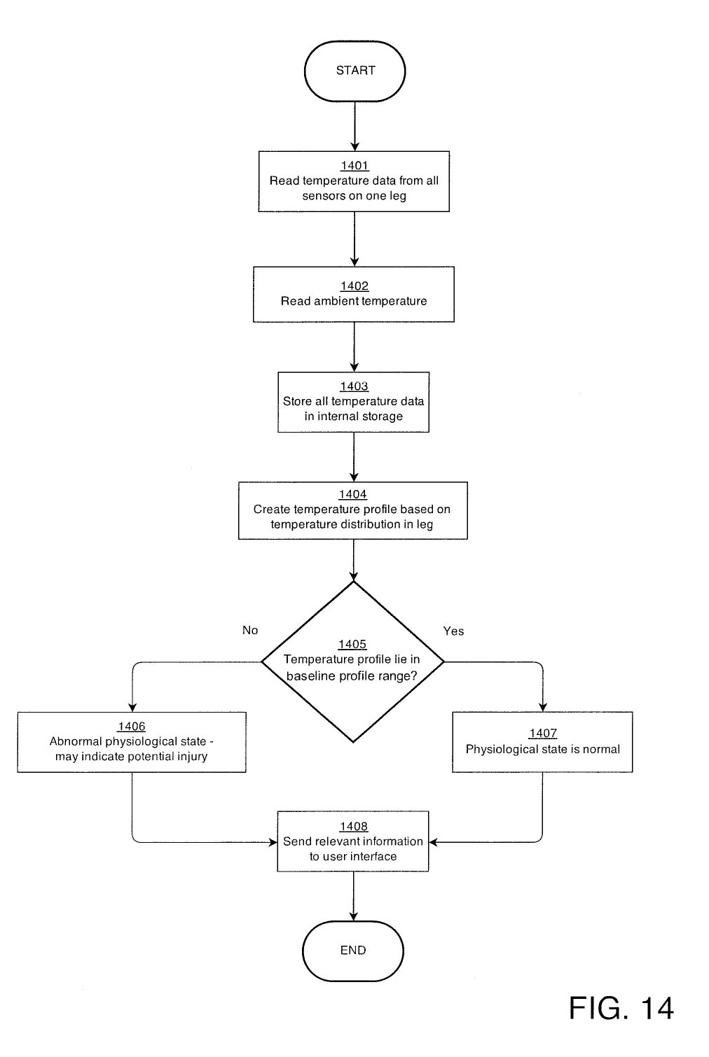

FIG. 14 shows an example of a process carried out by the display hub unit to determine whether or not the temperature profile of a leg is abnormal, which may be indicative of a potential injury. In block 1401, the temperatures from all sensors on one leg are read from the most recently received group of data. The ambient temperature may be read from the most recently received group of data in block 1402 and in block 1403 all data are stored. A temperature profile may be created in block 1404 based on the distribution of temperatures in the leg. In block 1405, this profile may be compared to the expected profile based on the baseline values. If the calculated profile matches the baseline profile, such as by exactly equaling or lying within a range of diagnostically-accepted deviations (which deviations may be configured based on the knowledge of one of ordinary skill of temperature variations that are normal or diagnostically-insignificant for horses, for a breed of horses, or a particular horse that are/is at rest) of the baseline profile, then the physiological state may be determined as “normal” as in block 1407. Otherwise, the physiological state may be determined as “abnormal” as in block 1406. Finally, in block 1408 the determined physiological state and relevant information may be sent to the user interface.

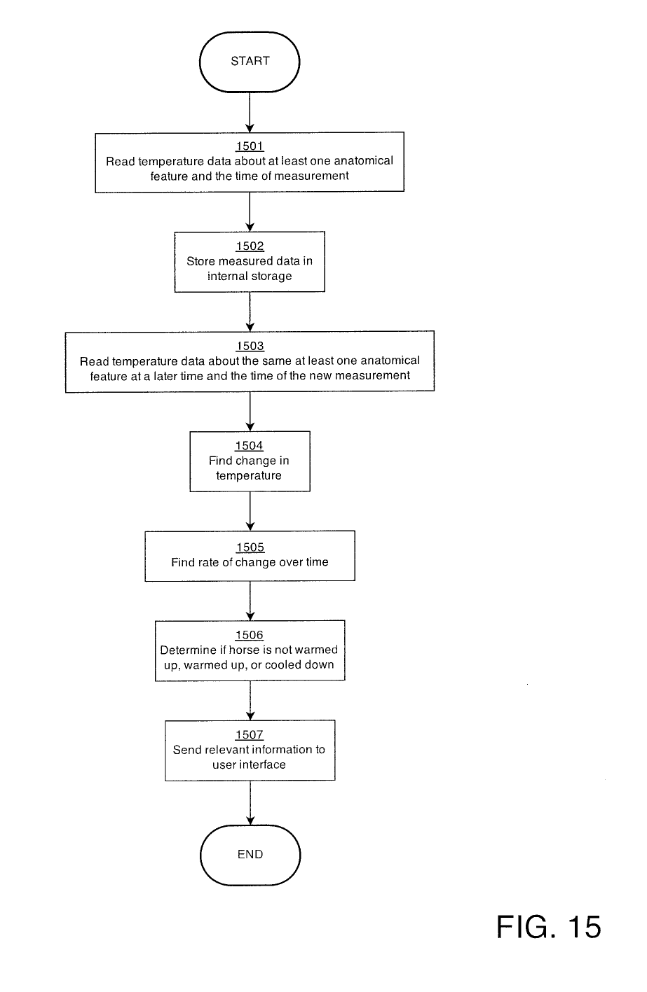

FIG. 15 shows an example of a process of determining a physiological state based on temperature differences with respect to time. In block 1501, temperature data for at least one anatomical feature may be obtained from one or more sensors by reading the most recently received group of data.. The time that data was measured is also read from the same group of data. In block 1502, that data are stored. In block 1503, which occurs at a later time, temperature about the same at least one anatomical feature is read from the even more recently received group of data. The time these data are received is also read. In block 1504, the difference between the temperature read in block 1503 and the temperature read in block 1501 is calculated. In block 1505, a rate of change is calculated by dividing the difference calculated in block 1504 by the difference in the times in which those temperatures were obtained. In block 1506, it is then determined whether the equine is not warmed-up, warmed-up, or cooled-down using a predetermined function that depends on the change in temperature from block 1504 and rate of change from block 1505. In block 1507, the resulting physiological state and relevant information is sent to the user interface.

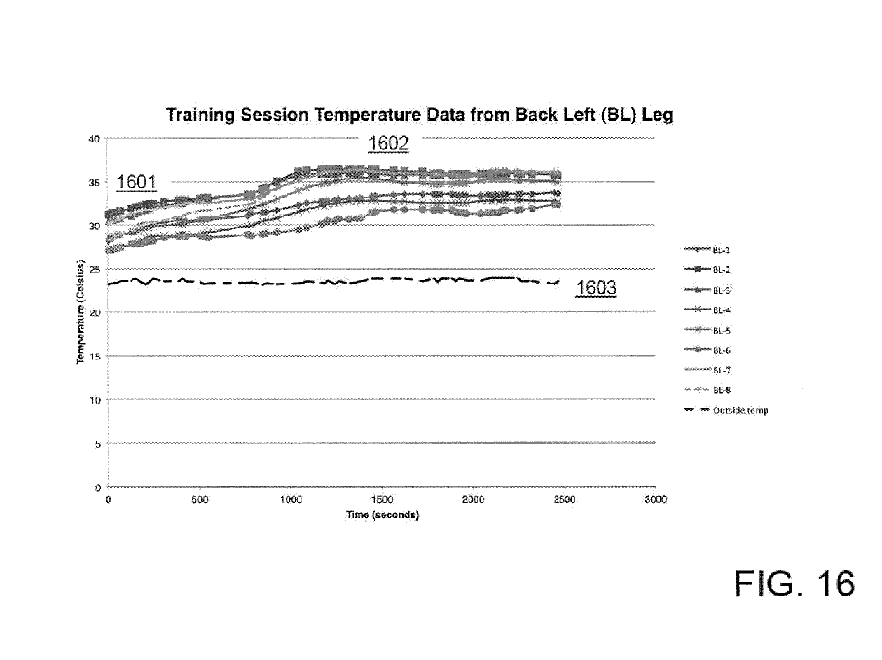

FIG. 16 shows an example of temperature data from the training session of a healthy equine in which eight sensors were placed at locations corresponding to anatomical features of interest and one sensor monitored the ambient temperature. An increase in temperature can be seen from the beginning 1601 of the session corresponding to the increased blood flow which is a result of exercise. A temperature plateau 1602 can also be seen which roughly correlates with the end of the warm up period, the point at which the display hub unit would alert the user as to the ‘warmed up’ physiological state, at which point moving to more intense exercise would be safe. The ambient temperature 1603 is also shown in this example.

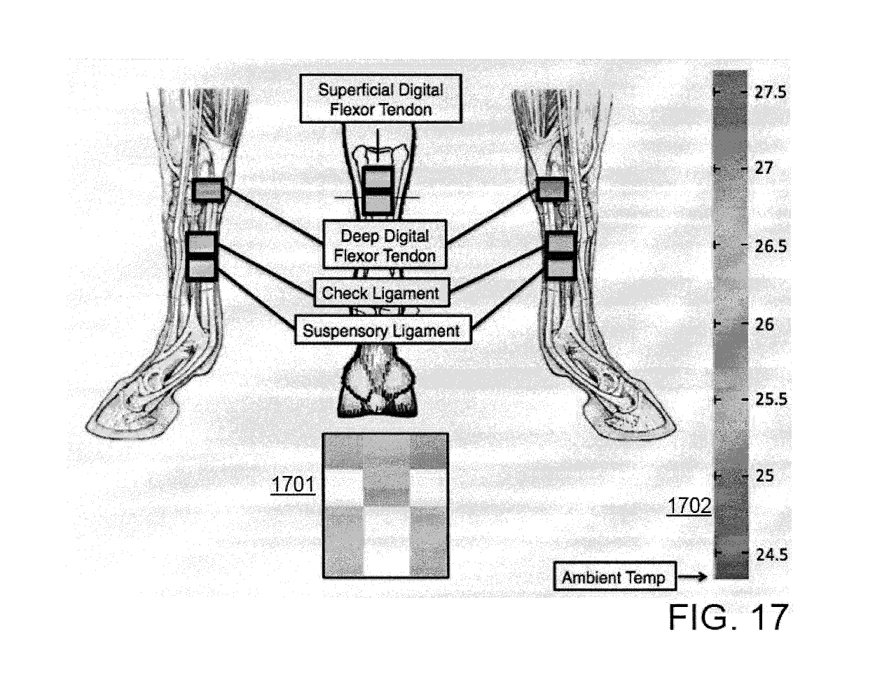

FIG. 17 shows an example of a temperature profile corresponding to the eight temperature sensors of FIG. 16. Each sensor is placed in a location that corresponds to a specific anatomical feature. The anatomical features in this example are labeled. The example visualization 1701 shows the temperature profile of the leg in which each area of the leg is represented as a block. The temperatures are shown in each block via a color that is chosen using a temperature-color scale 1702. The visualization 1701 is an example of what may be presented on the data visualization tool to user.

The embodiments and examples described above are focused on equines and the anatomical features in the equine's legs. This is done because of the prevalence of injuries to equines in this location and the anatomical structure of the leg. However, this invention may not be limited to this example. The wearable sensor unit may be used on other body parts of the equine where injury may occur. For example, back injuries are another common problem for competition equines. A possible embodiment of the wearable sensor unit may be a saddle blanket that covers the back area of the equine.

The monitoring system may also not be limited to equines. Other animals that compete may face similar injury problems and may have similar anatomical structures that may be monitored. The wearable sensor unit may be designed and constructed so that it may be comfortably worn by such an animal. Further, if the animal is not ridden then the display hub unit may not have to be worn by the user. In this case the data collection and visualization may occur on a local smart phone or tablet.

Techniques operating according to the principles described herein may be implemented in any suitable manner. Included in the discussion above are a series of flow charts showing the steps and acts of various processes that obtain, transmit, and analyze physiological information in the context of a physiological monitoring system for an equine. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non- persistent solid-state memory (e.g., Flash memory, Magnetic RAM, etc.), or any other suitable storage media. Such a computer-readable medium may be implemented in any suitable manner, including as a computer-readable storage media or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

A computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure, and are intended to be within the spirit and scope of the principles described herein. Accordingly, the foregoing description and drawings are by way of example only.

What is claimed is:

1. An apparatus to detect a temperature of at least one anatomical feature of an equine within a lower portion of a leg of the equine, the lower portion comprising a portion of the leg of the equine from a carpus or hock of the leg to a coffin joint of the leg of the equine, the apparatus comprising:
a housing having a shape conforming to a shape of at least a part of the lower portion of the leg of the equine and arranged to be removably attached to the lower portion of the leg of the equine to be worn by the equine; and
at least one temperature sensor integrated into the housing at one or more respective positions within the housing, wherein each one respective position of each one temperature sensor is a position that, when the apparatus is worn by the equine, corresponds to a position of one anatomical feature, of the at least one anatomical feature, within the lower portion of the leg of the equine and a temperature detected by the one temperature sensor is indicative of a temperature of the one anatomical feature to which the position of the one temperature sensor corresponds.

2. The apparatus of claim 1, wherein:
the housing is shaped to cover more than half of an area of the lower portion of the leg of the equine.

3. The apparatus of claim 1, wherein:
at least a first anatomical feature of the at least one anatomical feature is a connective tissue;
a first temperature sensor is disposed at a first position corresponding to a position of the connective tissue within the leg of the equine; and
the position of the connective tissue within the leg of the equine is a position at which, within the lower portion of the leg of the equine, the connective tissue is closest to an outer layer of skin of the leg of the equine.

4. The apparatus of claim 1, wherein:
the at least one temperature sensor is a plurality of temperature sensors;
the at least one anatomical feature is a plurality of anatomical features;
each of the plurality of anatomical features is a ligament or a tendon of the leg of the equine; and
each position of the at least one anatomical feature, to which a position of a temperature sensor of the plurality of temperature sensors corresponds, is a position at which, along a length of each one ligament or tendon within the lower portion of the leg of the equine, the one ligament or tendon is closest to an outer layer of skin of the equine.

5. The apparatus of claim 4, wherein a first temperature sensor and a second temperature sensor of the plurality of temperature sensors are integrated at positions corresponding to a position of a same anatomical feature of the plurality of anatomical features, with the first temperature sensor integrated at a position to be on one side of the leg of the equine and the second temperature sensor is integrated at a position to be on another side of the leg of the equine, when the apparatus is worn by the equine.

6. The apparatus of claim 4, wherein:
at least a portion of the housing is arranged to wrap around the leg of the equine;
the portion of the housing comprises an interior surface and an exterior surface, the interior surface being a surface to be closer to at least some skin of the equine than the exterior surface when the apparatus is wrapped around the leg of the equine;
at least some of the plurality of temperature sensors are positioned closer to the interior surface than to the exterior surface; and
the apparatus further comprises at least one second temperature sensor, disposed closer to the exterior surface than to the interior surface, to measure an ambient temperature.

7. The apparatus of claim 6, wherein:
each of the plurality of temperature sensors is configured to generate a signal indicative of a temperature at a position on the skin of the leg of the equine corresponding to the position at which, along a length of each one ligament or tendon within the lower portion of the leg of the equine, the one ligament or tendon is closest to an outer layer of skin of the lower portion; and
the apparatus further comprises:
at least one wireless transmitter; and
at least one control circuit configured to
generate temperature information based at least in part on output of the plurality of temperature sensors and the at least one second temperature sensor, and
operate the at least one wireless transmitter to transmit the temperature information.

8. A system comprising:
the apparatus of claim 7; and
a second apparatus comprising:
at least one wireless receiver;
at least one user interface; and
at least one second control circuit configured to, in response to receipt of the temperature information via the at least one wireless receiver, evaluate the temperature information to determine a physiological state and output the physiological state via the at least one user interface;
wherein the at least one control circuit of the apparatus is configured to repeatedly, according to a sampling interval, generate the temperature information and operate the at least one wireless transmitter to transmit the temperature information.

9. The system of claim 8, wherein the system comprises four of the apparatus, wherein two of the four apparatuses are adapted for a fore leg of the equine and two of the four apparatuses are adapted for a hind leg of the equine.

10. The apparatus of claim 7, further comprising:
an accelerometer; and
a heart rate sensor;
wherein the at least one control circuit is further configured to generate movement information based at least in part on output of the accelerometer, generate heart rate information based at least in part on output of the heart rate sensor, and operate the at least one wireless transmitter to transmit the movement information and the heart rate information.

11. An apparatus comprising:
at least one wireless receiver; and
at least one control circuit configured to:
receive via the at least one wireless receiver, over time, temperature information indicative of a temperature of at least one anatomical feature within a first leg of an equine;
evaluate the temperature information to determine a physiological state of the at least one anatomical feature within the first leg; and
output via a user interface an indication of the physiological state.

12. The apparatus of claim 11, wherein:
the at least one control circuit is configured to receive temperature information indicative of a temperature of at least one anatomical feature in each of four legs of the equine over time; and
the at least one control circuit is configured to evaluate the temperature information to determine a physiological state of each of the at least one anatomical features of each of the four legs of the equine.

13. The apparatus of claim 12, wherein:
the at least one control circuit is further configured to receive via the at least one wireless receiver heart rate information for the equine and/or information regarding movement of the equine; and
the at least one control circuit is further configured to determine an overall physiological state of the equine based at least in part on evaluating the physiological states of each of the at least one anatomical features of each of the four legs of the equine and evaluating the heart rate information and/or movement information.

14. The apparatus of claim 13, wherein the at least one control circuit is further configured to determine whether the equine has an overall physiological state that is one of a group of physiological states consisting of: not yet warmed up for exercise, warmed up for exercise, cooled down following being warmed up for exercise, and potentially injured.

15. The apparatus of claim 11, wherein:
the at least one control circuit is further configured to receive, via the at least one wireless receiver, ambient temperature information indicative of an ambient temperature during the time; and
evaluating the temperature information to determine the physiological state of the at least one anatomical feature of the first leg comprises evaluating the ambient temperature.

16. The apparatus of claim 11, wherein:
the at least one control circuit is further configured to determine a baseline state for each of the at least one anatomical feature of the first leg based at least in part on historical temperature information for the at least one anatomical feature of the first leg received over time; and
the at least one control circuit is further configured to evaluate the temperature information to determine the physiological state of the at least one anatomical feature of the first leg at least in part by comparing current temperature information for one or more of the at least one anatomical feature to the baseline state for the one or more of the at least one anatomical feature.

17. The apparatus of claim 16, wherein the at least one control circuit is further configured to, following a determination that at least a first anatomical feature, of the at least one anatomical feature of the first leg, is in a first physiological state:
receive, via the at least one wireless receiver, second temperature information indicative of the temperature of the at least one anatomical feature following the determination; and
evaluate the second temperature information to determine whether the first anatomical feature is in the baseline state, wherein evaluating the second temperature information comprises determining whether a current temperature of the first anatomical feature matches a temperature corresponding to the baseline state.

18. The apparatus of claim 11, wherein the at least one control circuit is further configured to evaluate the temperature information at least in part by determining a change in temperature of the at least one anatomical feature over the time, wherein determining the change comprises comparing current temperature information and prior temperature information for the at least one anatomical feature during the time.

19. The apparatus of claim 11, wherein the at least one control circuit is configured to evaluate the temperature information at least in part by determining whether a current temperature of a first anatomical feature of the at least one anatomical feature is greater than a threshold temperature value.

20. The apparatus of claim 11, wherein:
the at least one anatomical feature comprises a first anatomical feature of the first leg of the equine;
the equine has a second leg that is bilaterally symmetric to the first leg of the equine and includes a second anatomical feature that is bilaterally symmetric to the first anatomical feature; and
the at least one control circuit is further configured to receive via the at least one wireless receiver, over the time, second temperature information indicative of a temperature of the second anatomical feature of the second leg of the equine; and
the at least one control circuit is configured to evaluate the temperature information to determine the physiological state of the first anatomical feature of the first leg at least in part by comparing the temperature of the first anatomical feature indicated by the temperature information for the first anatomical feature and the temperature of the second anatomical feature indicated by temperature information for the second anatomical feature.
